(19) United States
(12) Patent Application Publication
(10) Pub. No.: US 2003/0167021 A1
(43) Pub. Date: Sep. 4, 2003

(54) Apparatus for locating and anesthetizing nerve groups

(76) Inventor: Peter B. Shimm, Chevy Chase, MD (US)

Correspondence Address: PILLSBURY WINTHROP, LLP, P.O. BOX 10500, MCLEAN, VA, 22102, US

(21) Appl. No.: 10/086,441

(22) Filed: Mar. 4, 2002

(51) Int. Cl. A61B 5/05

(52) U.S. Cl. 600/554

(57) ABSTRACT

An apparatus for locating and anesthetizing a nerve enables an anesthesiologist to perform a nerve blockade procedure without an assistant. An anesthetic-filled syringe is releasably mounted to a hands-free, foot-actuated, hydraulic syringe controller. An electric current generator is mounted to the syringe controller and operatively connected to a needle of the syringe. A hands-free, foot-actuated current generator controller determines an amperage of an electrical stimulus output to the needle. The current generator includes audible and visual displays for informing the anesthesiologist of the output amperage. Because the syringe controller and current generator controller allow hands-free operation, an anesthesiologist can continuously use both hands to manipulate and hold the needle in a patient.

### BACKGROUND OF THE INVENTION

1. Field of the Invention

The present invention relates generally to an apparatus for locating and anesthetizing nerve groups.

2. Description of Related Art

The practice of regional anesthesia, the administration of anesthesia to a specific body region, is entering a renaissance. An increasing number of patients are receiving anesthetic nerve blocks during surgery, for the relief of post-operative pain, and for the extended relief of chronic pain. Numerous studies have shown that regional anesthesia is often preferable to the use of general anesthesia because of increased safety and patient satisfaction, excellent postoperative pain control, and a decrease in anesthesia costs.

Through regional anesthesia, a bolus of local anesthetic is delivered into close contact with a peripheral nerve, nerve group, or nerve plexus, thereby enabling neuronal blockade to occur. In the discussion that follows, reference to a peripheral nerve shall be understood also to refer to a nerve group or nerve plexus. Typically, a syringe containing a solution of local anesthetic, with a needle attached to it, is utilized to perform the blockade of a peripheral nerve. Because the anesthetic bolus must be delivered near the nerve to be blocked, various methods have been developed to ensure that the needle is adjacent to the nerve before the bolus is injected.

Peripheral nerve stimulation (PNS) provides one conventional method for ensuring that the needle is in close proximity to the target nerve. The practice of nerve localization via electrical stimulation relies on the fact that an electrical pulse can stimulate a motor nerve fiber to contract an innervated muscle or cause paresthesia in the case of sensory nerve stimulation.

When localizing a nerve using a nerve stimulator, an electrified anesthesia needle having a current of approximately 1.5-3.0 mA is placed within the tissue of the body in the vicinity of the nerve to be blocked. The needle is then slowly advanced as a stimulating probe until stimulation of the target nerve is achieved, as determined by visually detecting muscle contractions or by eliciting a report that the patient feels the stimulus. Once the expected response is observed, the current is gradually decreased as the needle is moved closer to the nerve, until nerve stimulation is obtained using a lower amperage current. Typically, a response at 0.2-0.5 mA assures that the tip of the needle is in close proximity to the nerve, therefore providing a reliable nerve blockade with injection of the local anesthetic.

Once a response has been observed in the 0.2-0.5 mA range, a small portion of the anesthetic dose is administered to the patient as a test dose to terminate the response of the nerve to the electrical pulse. The output current is then once again increased to assure that the cessation of the response is a result of the nerve blockade, rather than unintentional repositioning of the needle away from the nerve. If a nerve response is still absent after the output current has been increased, the anesthesia needle is deemed to be in the vicinity of the target nerve and the remaining bolus of local anesthetic solution is injected.

The same technique can be employed regardless of whether the nerve to be localized is motor or sensory. A description of this nerve localization technique is discussed in greater detail in Raj et al., “Use of the Nerve Stimulator for Peripheral Blocks”, Regional Anesthesia, April-June 1980, pp. 14-21.

Examples of nerve stimulators for assisting in the administration of anesthesia may be found in U.S. Pat. No. 3,682,162 to Coyler and U.S. Pat. No. 4,515,168 to Chester et al. The Coyler patent generally describes a combined electrode and syringe needle, which acts as a stimulation probe when the syringe needle is connected to an electrical supply. The Chester et al. patent discloses a nerve stimulator that is clamped onto the syringe of a conventional syringe and anesthesia needle assembly. The unit contains a power supply, a pulse generating circuit, and a manually controlled current-adjusting potentiometer, which allows the operator to adjust the current supplied to the needle.

To understand the problem associated with the prior art, it is first necessary to understand the nerve blockade procedure as it is performed today. The blockade procedure involves four steps. First, with one hand, the anesthesiologist palpates the landmarks on the patient and draws the skin taught between the index and middle fingers. This first step prepares the location on the patient's body for the anesthesia needle. Second, with his other hand, the anesthesiologist holds the needle and guides it into the patient at the appropriate location in the area palpated. Third, an assistant is required to control the syringe, either to aspirate (suck in) fluid or inject (push out) fluid. Fourth, the assistant is required to man the nerve stimulator so that the anesthesiologist may determine the optimal location for delivery of the anesthesia bolus to the patient.

As indicated above, this four-step procedure currently may be performed only with two people working together. The anesthesiologist is responsible for the first and second steps. The assistant performs the third and fourth steps. It is unfeasible for a single anesthesiologist to perform all four steps because the anesthesiologist must use both hands to position the needle and keep it stationary once the nerve is located. The anesthesiologist, therefore, does not have a free hand for operating the syringe or the current applied by the nerve stimulator.

As a result, an assistant is required to perform the nerve blockade procedure. The required assistant may be one selected from a group of medical practitioners comprising surgeons, scrub technicians, nurses, and a second anesthesiologist.

The need for an assistant significantly increases the cost and complexity of performing nerve blockades using the PNS technique. One reason for this is the fact that the same assistant is not always available to the anesthesiologist. Accordingly, the level of assistance that the assistant provides may vary significantly from one assistant to another. Consequently, the anesthesiologist must frequently spend considerable amounts of time discussing the procedure with the assistant or providing on-the-spot training to ensure that the procedure is completely effective. Further, the need for continuous communication between the anesthesiologist and assistant during the procedure complicates the procedure and may interfere with other aspects of the surgery.

U.S. Pat. No. 5,830,151 to Hadzic et al. (the '151 patent), which is incorporated herein by reference in its entirety, discloses an apparatus that simplifies the PNS technique by enabling a single person to perform three of the four necessary steps. The '151 patent discloses a foot-pedal-operated nerve stimulator attached to a hand-operated syringe. The foot pedal enables the anesthesiologist to control and modify, within a selected current range (measured in milliamps (mA)), the magnitude of the electrical stimulus. Use of the foot pedal to perform step (4) leaves both of the anesthesiologist's hands free to perform steps (1) and (2). Unfortunately, an assistant is still needed to perform step 3 (controlling the syringe).

U.S. Pat. No. 5,284,153 to Raymond et al. discloses a device that performs step (4) automatically. The disclosed device includes an anesthesia needle coupled to an electrical source and a device for detecting nerve response to the electrical stimuli. The amount of current generated by the electrical source is automatically controlled so as to maintain the signal generated as a function of the response of the nerve to the stimuli. The closer the stimulating needle comes to the nerve, the higher the detected response is, which in turn automatically decreases the electrical stimulus. While step (4) is automatically performed, an assistant is still needed to perform step (3).

While locating the target nerve, constant aspirating pressure should be applied to the syringe (by pulling on the syringe plunger) to develop a suction at the tip of the needle. If the anesthesiologist finds that the syringe is aspirating blood (by observing the inflow of blood into the syringe), it is likely that the tip of the needle has entered a vein or artery. If so, the position of the needle must be changed before the anesthetic may be applied to the desired nerve plexus. Otherwise, the injected anesthetic might flow away from the target nerve and be ineffective (or at least significantly less effective).

The flow rate of the aspiration (or the pressure applied to aspirate the syringe) is not critical to performing the nerve blockade procedure. Aspiration is only needed to the extent necessary to assure that blood (or other bodily fluids) will be drawn into the syringe should the needle enter a fluid-containing body area. Such aspiration is needed because venous pressure typically is 0-5 mm Hg. Without aspiration, venous pressure would not be insufficient, in and of itself, to force blood into the syringe and provide any indication that the tip of the needle is improperly positioned in the patient. This is less important should the needle pierce an artery because arterial pressure is generally 120/80 mm Hg, which is sufficient to push blood into the syringe under most circumstances. However, even in the case of an artery, it is preferred to have an aspirating pressure applied to the syringe.

In order to apply requisite aspiration pressure to the syringe while the target nerve plexus is being located, an assistant must apply constant aspirating pressure to the syringe. This procedure constantly occupies at least one of the assistant's hands, is physically demanding, and requires the assistant to continuously think about applying the aspirating pressure.

Various conventional automatically controlled syringes are known in the art. U.S. Pat. No. 5,584,814 to Schuster et al. discloses a hydraulically-powered syringe automation system. PCT Application No. PCT/US88/01644 discloses a remotely-controlled, electric-motor-driven aspirating hypodermic syringe.

While the prior art has addressed various individual steps in the nerve blockade procedure, no single prior art reference describes an apparatus that permits the anesthesiologist to perform a neural blockade without an assistant. This deficiency has created a need that has been, heretofore, unfulfilled.

### SUMMARY OF THE INVENTION

One aspect of the present invention, therefore, is to provide an apparatus that addresses the deficiencies in the prior art.

One aspect of the present invention provides an improved apparatus for locating and anesthetizing nerve groups.

An additional aspect of the present invention provides an apparatus for locating and anesthetizing nerves that is cost-efficient to use.

A further aspect of the present invention provides a compact, easily transportable apparatus for locating and anesthetizing nerves.

A further additional aspect of the present invention provides an apparatus that enables a single person to efficiently locate and anesthetize nerves.

A further additional aspect of the present invention provides an apparatus for locating and anesthetizing nerves that includes a needle adapted to be connected to a syringe having an anesthetic therein. An electric current generator operatively connects to the needle to selectively apply an electrical stimulus to the needle. A hands-free current generator controller operatively connects to the current generator. The current generator controller determines electrical characteristics, including at least an amperage, of the electrical stimulus. A hands-free syringe controller is adapted to be connected to the syringe to selectively aspirate the syringe and inject the anesthetic through the needle.

The syringe controller and/or the current generator controller may be foot-operated.

According to a further aspect of the present invention, the syringe controller comprises a foot pedal movably connected to a base for movement relative to the base between compressed and uncompressed positions. A master hydraulic cylinder has a first cylinder portion mounted to one of the foot pedal and the base and a first piston portion mounted to the other of the foot pedal and the base. The first cylinder portion and first piston portion slidingly engage each other such that compression of the foot pedal contracts the master cylinder. A slave hydraulic cylinder has a second cylinder portion adapted to be mounted to one of a syringe base and plunger and a second piston portion adapted to be mounted to the other of the syringe base and plunger. The second cylinder portion and second piston portion slidingly engage each other. A first fluid pathway hydraulically connects the master cylinder to the slave cylinder such that contraction of the master cylinder extends the slave cylinder, which, in turn, is adapted to contract the syringe and inject the anesthetic through the needle.

The syringe controller may be constructed to releasably secure the syringe thereto.

The current generator may include a speaker that calls out audibly the amperage being output by the current generator.

The current generator may further include a light that flashes each time the electric stimulus is applied to the needle.

A further aspect of the present invention provides a foot-actuated syringe controller having a syringe that includes a syringe base and a plunger. The syringe controller also includes a foot pedal base. The syringe controller further includes a foot pedal movably connected to the foot pedal base. The foot pedal has actuated and unactuated positions relative to the foot pedal base and is operatively connected to the syringe. Moving the foot pedal into the actuated position moves the plunger toward the syringe base, thereby injecting a fluid out of the syringe. Releasing the foot pedal into the unactuated position applies a force to the plunger that tends to move the plunger away from the syringe base, thereby aspirating the syringe.

Additional and/or alternative objects, features, aspects, and advantages of the present invention will become apparent from the following description, the accompanying drawings, and the appended claims.

### BRIEF DESCRIPTION OF THE DRAWINGS

For a better understanding of the present invention as well as other objects and further features thereof, reference is made to the following description which is to be used in conjunction with the accompanying drawings, where:

FIG. 1 is a schematic diagram of an apparatus according to the present invention;

FIG. 2 is a partial side view of a portion of the apparatus illustrated in FIG. 1; and

FIG. 3 is a partial front view of the portion of the apparatus illustrated in FIG. 2.

### DETAILED DESCRIPTION OF PREFERRED EMBODIMENTS OF THE INVENTION

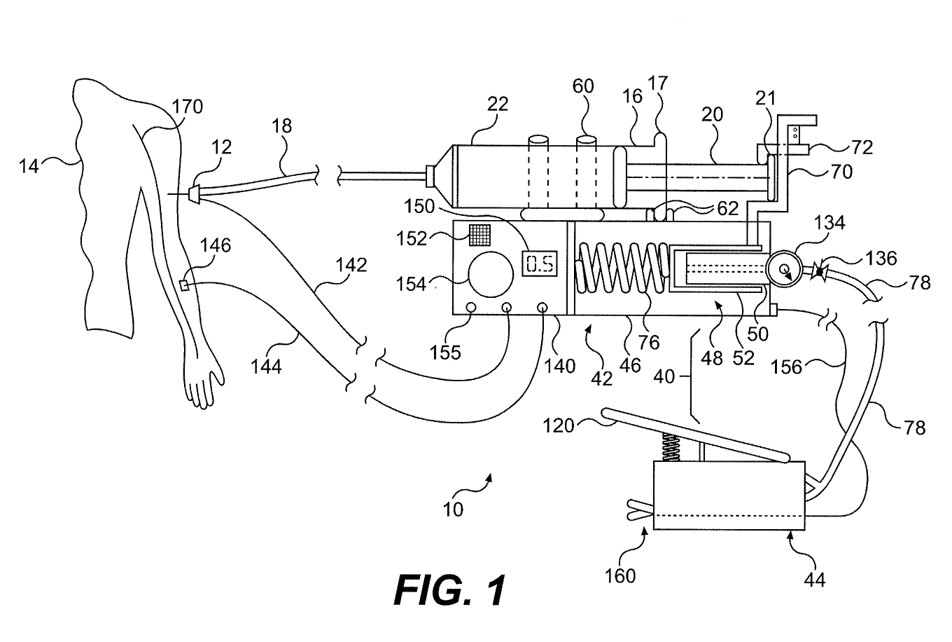

FIG. 1 is a schematic view of an apparatus 10 for locating and anesthetizing a nerve. A hypodermic needle 12 having an electrode is adapted to be inserted by an anesthesiologist into the tissue of a patient 14. The needle 12 is connected to a syringe 16 via a flexible tube 18. The syringe 16 is of a standard type and includes a plunger 20 slidably fit into a syringe cylinder (or base) 22. While any size or type of syringe may be employed, the syringe 16 illustrated is preferably a 60 cc syringe.

As described below, the syringe 16 is removably mounted onto a hands-free syringe controller 40. The syringe controller 40 includes a syringe mounting assembly 42 and a base 44.

The syringe mounting assembly 42 comprises a frame 46 with a slave hydraulic cylinder 48 mounted therein. The slave cylinder 48 includes a piston portion 50 slidably fit into a cylinder portion 52 to define an enclosed slave cylinder volume. A seal (not shown) is disposed between the cylinder portion 52 and the piston portion 50 to seal the enclosed slave cylinder volume. The piston portion 50 is rigidly mounted within the frame 46. The cylinder portion 52 is movably mounted within the frame 46 so that is slides relative to the piston portion 50.

A syringe base clamp 60 is mounted onto the frame 46. The base clamp 60 removably secures the syringe 16 to the frame 46. The base clamp 60 also immovably secures the syringe 16 relative to the movable cylinder portion 52.

As illustrated, the frame 46 preferably includes two ridges 62 that are designed to fit around a radially-extending handle portion 17 of the syringe base 22 to prevent the syringe base 22 from moving relative to the frame 46 when clamped into position. While the two ridges 62 are shown as a part of the frame 46, the two ridges 62 may be incorporated as a part of the base clamp 60.

A plunger clamp 70 is mounted to the cylinder portion 52 of the slave cylinder 48. The plunger clamp 70 moves with the cylinder portion 52 relative to the piston portion 50 and frame 46. The plunger clamp 70 includes a spring-loaded lever 72 that releasably secures an enlarged head 21 of the plunger 20 to the plunger clamp 70. Because the syringe base clamp 60 and plunger clamp 70 are designed to releasably secure any standard syringe 16, the apparatus 10 is versatile.

When the syringe 16 is secured in the base clamp 60 so that the enlarged head 21 is captured by the plunger clamp 70, the plunger 20 moves together with the cylinder portion 52 of the slave cylinder 48. Accordingly, when the cylinder portion 52 moves away from the piston portion 50, the slave cylinder 48 expands and the plunger 20 is forced into the syringe 16, thereby forcing fluid from the syringe 16. In this manner, anesthetic in the syringe 16 is forced through the needle 12. Alternatively, when the slave cylinder 48 contracts, the plunger 20 is withdrawn from the syringe 16 to aspirate fluid into the syringe 16 through the needle 12.

A resilient member 76 is operatively connected between the cylinder portion 52 and the frame 46. The resilient member 76 applies a force onto the cylinder portion 52 to force the cylinder portion 52 towards the piston portion 50 of the slave cylinder. In other words, the resilient member 76 applies a contracting force to the slave cylinder 48 that causes the slave cylinder 48 to contract and, thereby, aspirate the syringe 16.

In the embodiment illustrated, the resilient member 76 comprises a compression spring connected at one end to the cylinder portion 52. The opposite end of the resilient member 76 is connected to the frame 46.

An input/output conduit 78 is operatively connected at one end to the slave cylinder 48 via a connection to the piston portion 50. As illustrated in dotted lines in FIG. 1, the conduit 78 extends through the slave piston portion 50 to the inside (slave cylinder chamber) of the slave cylinder 48. An opposite end of the input/output conduit 78 is connected to the base 44. The input/output conduit 78 preferably comprises a high-pressure flexible tube.

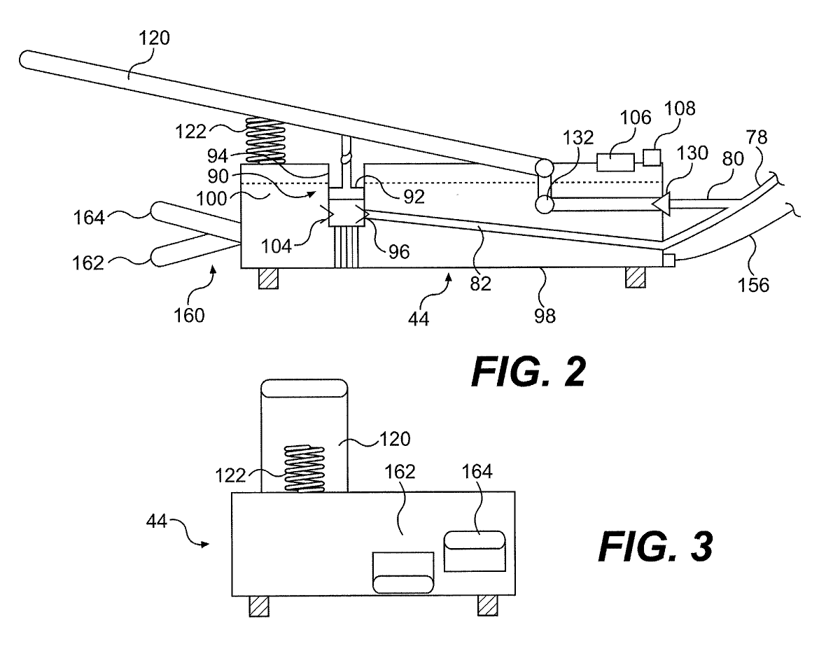

Hereinafter, the base 44 will be described with reference to FIGS. 2 and 3. The input/output conduit 78 diverges at the base into an input conduit 80 and an output conduit 82.

The output conduit 82 operatively connects to a master cylinder 90 that includes a master piston portion 92 that slidably engages a master cylinder portion 94 to define a master cylinder volume. A seal (not shown) is disposed between the master cylinder portion 94 and the master piston portion 92 to seal the enclosed master cylinder volume. The master cylinder 90 and slave cylinder 48 are hydraulically linked via a first pathway defined by the output conduit 82 and the input/output conduit 78 such that contraction of the master cylinder 90 extends the slave cylinder 48 and contracts the syringe 16.

A one-way check valve 96 is disposed within the output conduit 82. The check valve 96 allows fluid to flow only through the output conduit 82 and first pathway toward the slave cylinder 48.

The master cylinder portion 94 is rigidly mounted within a base frame 98.

A fluid reservoir 100 is supported by and/or incorporated into the base frame 98. Because the reservoir 100 is supported by the base frame 98, the weight of the fluid therein stabilizes the base 44 and reduces the overall size of the apparatus 10. The slave cylinder 48 is operatively connected to the reservoir 100 via a second pathway defined by the input/output conduit 78 and the input conduit 80, which is operatively connected to the reservoir 100. The master cylinder 90 is operatively connected to the reservoir 100 via a third pathway that includes a check valve 104 that allows fluid to flow only from the reservoir 100 toward the master cylinder 90. An air pressure valve 106 connects the reservoir 100 to the ambient atmosphere to allow varying levels of fluid in the reservoir 100.

A drain 108 connects the reservoir 100 to the ambient atmosphere and facilitates filling and emptying of the reservoir 100. The drain 108 preferably comprises a manual valve or a stopcock. The fluid used in the hydraulic circuit of the syringe controller 40 may be water, hydraulic fluid, oil, or other incompressible fluid.

If water is chosen as the hydraulic circuit fluid, the reservoir 100 can be easily drained before being transported. The reservoir 100 can then be refilled before the apparatus 10 is used at a new location. The ability to empty the hydraulic circuit and reservoir 100 of the apparatus 10 prior to transporting the apparatus 10 to a new location reduces the transportation weight of the apparatus 10 and enhances transportability.

A foot pedal 120 is movably connected to the base frame 98. The foot pedal 120 has compressed/actuated (downward in the illustrated embodiment) and uncompressed/unactuated (upward in the illustrated embodiment) positions relative to the base frame 98. In the illustrated embodiment, the foot pedal 120 is pivotally connected to the base frame 98. A resilient member 122 is connected between the foot pedal 120 and the base frame 98 and tends to force the foot pedal 120 into the uncompressed position. In the illustrated embodiment, the resilient member 122 comprises a compression spring. The foot pedal 120 is connected to the master piston 92 such that compression of the foot pedal 120 contracts the master cylinder 90. In the illustrated embodiment, the master piston 92 is hingedly connected to the foot pedal 120 to allow for slight rotational movement of the foot pedal 120 relative to the master piston portion 92 as the foot pedal 120 pivots relative to the base frame 98.

A check valve 130 is disposed in the output conduit 80 of the second fluid pathway. The check valve 130 allows fluid to flow only through the output conduit 80 toward the reservoir 100. A valve 132 is also disposed in the output conduit 80 in series with the check valve 130. The valve 132 is controlled by the foot pedal 120 such that the valve 132 is only open when the foot pedal 120 is in the uncompressed position. When the foot pedal 120 is compressed into any of a plurality of compressed positions, the valve 132 closes and prohibits fluid from flowing from the slave cylinder 48 through the second pathway and output conduit 80 toward and into the reservoir 100.

A pressure gauge 134 is preferably operatively connected to the input/output conduit 78 at or near the syringe mounting assembly 42 so that the anesthesiologist can monitor the pressure being applied to the syringe 16. A pressure relief valve (or pop-off valve) 136 is also preferably operatively connected to the input/output conduit 78 and set such that the relief valve 136 opens when a pressure in the input/output conduit 78 (and consequently the slave cylinder 48 and syringe 16) exceeds a predetermined pressure. The relief valve 136 ensures that the syringe 16 and needle 12 are never over-pressurized.

While the illustrated syringe controller 40 is hydraulically powered, the present invention is not so limited. However, while other controllers may be used (e.g. pneumatic or electromechanical (i.e. with a motor and screw or with a stepper motor)), the hydraulic system is preferred for a number of reasons.

First, the types of syringes 16 that are normally used to deliver anesthesia typically have a large bore diameter. As a result, a considerable amount of force is required to deliver the required amount of anesthesia to the patient 14. The illustrated foot-actuated hydraulic syringe controller 40 improves control over the syringe 16 relative to other controllers because the anesthesiologist can apply the force of his/her entire weight to the foot pedal 120 to inject the anesthesia. The syringe controller 40 can also be designed to provide a large mechanical advantage to the foot pedal (for example by positioning the master cylinder 90 near the pivot point between the foot pedal 120 and the base frame 98 or by reducing the diameter of the master cylinder 90 relative to the slave cylinder 48). Consequently, the weight of the anesthesiologist is expected to provide more than a sufficient force to inject the anesthesia into the patient 14.

Second, a hydraulic system such as the one illustrated as the syringe controller 40 is preferred because the anesthesiologist has a “feel” for the pressure being applied by the syringe 16 and also has a “feel” for the operation of the apparatus 10. This “feel” exists because any resistance to injection experienced by the syringe 16 is transferred to the foot pedal 120, via the hydraulic circuit, such that the foot pedal 120 resists compression as well. If the syringe 16 is pneumatically or electro-mechanically controlled, the anesthesiologist loses this sense of “feel” for the injection operation. Accordingly, it is possible that the anesthesiologist may over-inject or under-inject. The proper “feel” for the injection operation is also important because the anesthesiologist is usually providing very fine control over the injection process. Only a small amount of anesthesia may be needed, depending upon the area where the anesthesia is to be delivered. This also depends on the nerve group that the anesthesiologist is trying to block with the anesthesia.

Third, a hydraulic system such as the illustrated syringe controller 40 is far more compact than a pneumatic or electromechanical system. In a pneumatic system, a large cylinder of pressurized gas is needed to inject the anesthesia. The bulk and weight of large gas cylinders greatly reduces the transportability of the apparatus. Similarly, electromechanical systems are often large because the motors that apply pressure to the plunger 20 on the syringe 16 must be large enough to deliver the force needed to inject the anesthesia. Further, the motors are quite heavy.

Fourth, electromechanical systems that use motors and screw-type apparatuses are very slow-moving. It is difficult for the anesthesiologist and/or the patient to hold still for a long enough period of time to complete an injection using such a system. Faster injection systems such as the illustrated hydraulic syringe controller 40 are therefore preferred.

Hereinafter, a current generator (or nerve stimulator) 140 will be described with reference to FIG. 1. The current generator 140 may be any type of conventional current generator that would be known to one skilled in the art to be used for nerve stimulation. The generator 140 preferably generates a 1 Hz DC pulse at an amperage determined by a controller. As would be appreciated by one skilled in the art, however, other frequencies may also be used.

The current generator 140 is preferably incorporated into the syringe mounting assembly 42 to make the apparatus 10 more compact. A negative output lead 142 of the current generator is connected to the electrode of the needle 12. A positive output lead 144 is connected to an electrode of a grounding pad (or ECG electrode) 146 that is adapted to be attached to the skin of the patient 14. When the needle 12 is inserted into the patient 14 and the grounding pad 146 is attached to the skin of the patient 14, an electrical circuit extending from the first lead 142 to the needle 12, from the needle 12 to the patient 14, from the patient 14 to the grounding pad 146, and from the grounding pad 146 to the second lead 144 is formed.

The current generator 140 preferably includes a variety of auditory and visual displays that provide the anesthesiologist with information about the electrical stimulus being applied to the needle 12. The current generator preferably includes a visual display 150 that illustrates an amperage of the current/electrical stimulus being output to the leads 142, 144. The current generator 140 also preferably includes a speaker 152 that calls out audibly the amperage being output by the current generator 140. The current generator 140 further preferably includes a manual switch 154 that allows a user to manually adjust the current or the current range of the current generator 140. The current generator further preferably includes an LED (or other type of light) 155 that flashes each time an electrical stimulus is output to the needle 12. The flashing LED 155 provides an indication to the anesthesiologist that the current is being applied to the patient, which may be necessary if the current is not creating the expected nerve response in the patient 14. Alternatively, the flashing LED 155 may be relied upon to reassure the anesthesiologist that the nerve block has been successfully completed.

The current generator 140 is controlled by a hands-free current generator controller 160 that is operatively connected to the current generator 140 via a current control lead 156, which preferably includes a plurality of control wires. The generator controller 160 determines electrical characteristics, including at least an amperage, of the electrical stimulus output to the leads 142, 144.

In the illustrated embodiment, the hands-free current controller 160 is foot-controlled and is incorporated into the base 44 to make the apparatus 10 more compact. The controller 160 includes foot-actuated electrical switches 162, 164. The switches 162, 164 are preferably momentary switches. To illustrate both the depressed/actuated and released/unactuated positions of the electrical switches 162, 164 in FIGS. 1-3, the electrical switch 162 is shown in its actuated/depressed position (i.e., its position when the anesthesiologist's foot (not shown) is pushing down the electrical switch 162), while the electrical switch 164 is shown in its unactuated/released position. The generator controller 160 and current generator 140 are designed such that repeated actuation of the first switch 162 incrementally increases the amperage of the electrical stimulus. Conversely, repeated actuation of the second switch 164 incrementally decreases the amperage of the electrical stimulus. The controller 160 may also be designed such that continuous actuation of either switch 162, 164 adjusts the amperage by more than one increment. The incremental amperage change per tap of the switches 162, 164 is preferably about 0.1 mA. The generator 140 and controller 160 are further designed such that when the generator 140 is off, actuation of either switch 162, 164 turns on the generator 140.

Hereinafter, operation of the apparatus 10 will be described. The syringe 16 is filled with anesthetic and clamped onto the slave cylinder 48. The syringe mounting assembly 42 is then placed in an area that is readily viewable by the anesthesiologist. Because the connections, leads, and conduits 18, 78, 142, 144, 156 between the needle 12 and the syringe mounting assembly 42 and between the syringe mounting assembly 42 and the base 44 are long and flexible, the anesthesiologist may place the syringe mounting assembly 42 in any convenient location without interfering with the functionality of the apparatus 10. The grounding pad 146 is then connected to the skin of the patient 14.

Next, the anesthesiologist compresses the uncompressed foot pedal 120 with his/her foot. This action closes the valve 132 and contracts the master cylinder 90. Contraction of the master cylinder 90 forces fluid through the conduits 82, 78 and into the slave cylinder 48, thereby extending the slave cylinder 48, contracting the syringe 16 slightly, and flushing any air out of the tube 18 and needle 12.

The anesthesiologist keeps the foot pedal 120 at least slightly compressed so that the valve 132 remains closed and fluid is prevented from flowing out of the slave cylinder 48 into the reservoir via the conduits 78, 80. Consequently, the slave cylinder 48 cannot contract, despite the contracting force being applied to the slave cylinder 48 by the resilient member 76.

The anesthesiologist then uses one hand to palpate landmarks on the patient 14 near the target nerve 170 and inserts the needle 12 into the patient 14 with his/her other hand. Because the location of the needle 12 in the patient 14 is critical, the anesthesiologist uses both hands to palpate landmarks, compress and hold the skin taut, and manipulate and immobilize the needle 12 throughout the entire nerve anesthetization procedure.

Once the needle 12 is inserted into the patient 14, the anesthesiologist releases the foot pedal 120, allowing the resilient member 122 to push the foot pedal 120 into the uncompressed position. The valve 132 is thereby opened, allowing fluid to flow from the slave cylinder 48 to the reservoir via the conduits 78, 80 and allowing the resilient member 76 to apply a contracting pressure to the slave cylinder 48, which aspirates the syringe 16. The automatic aspiration continues until the anesthesiologist recompresses the foot pedal 120. The apparatus 10 therefore greatly simplifies the procedure relative to conventional apparatuses, which require the anesthesiologist or assistant to manually apply continuous suction to the syringe 16.

If the current generator 140 is not already on, the anesthesiologist next turns it on by tapping on either switch 162, 164 with his/her foot. Using the foot switches 162, 164, the anesthesiologist preferably sets the initial amperage to between 1.5 and 2.0 milliamps (mA). At this amperage the anesthesiologist expects to see a twitch in the patient 14 to show that the needle 12 is near the target nerve 170 (or nerve group or nerve plexus) that is to be blocked. As the anesthesiologist moves the needle 12 closer and closer to the nerve 170, he/she reduces the amperage applied to the needle 12 by tapping on the switch 164 with his/her foot until the nerve twitch is extinguished. The needle 12 is then moved until the twitch returns at the lower amperage. This procedure is repeated until the anesthesiologist observes a twitch with an amperage of 0.5 mA or less.

Throughout the nerve locating step, it is important that the anesthesiologist know the amperage currently being applied to the needle 12. Because the syringe mounting assembly 42 is positioned in a convenient place, the anesthesiologist can quickly glance at the display 150 to determine the instantaneous amperage. Preferably, however, the anesthesiologist need only listen to the amperage being called out by the speaker 152. By relying on the speaker 152 instead of the display 150, the anesthesiologist is less likely to accidentally reposition the needle 12 than if the anesthesiologist has to turn his/her head to view the display 150. However, proper positioning of the syringe mounting assembly 42 and display 150 minimizes the risk.

Throughout the locating step, the anesthesiologist monitors the tube 18 and/or the syringe 16. If the anesthesiologist observes blood or other bodily fluid being aspirated, he/she learns that the needle 12 is not positioned properly and can reposition the needle 12 to avoid delivering the anesthetic ineffectively into a vein instead of near the target nerve 170.

Once the anesthesiologist has positioned the needle 12 in a position where blood/bodily fluid is not being aspirated and the twitch is present when the amperage is 0.5 mA or less, the anesthesiologist is confident that application of the anesthesia will be nearly (almost 100%) effective in blocking pain. To inject the bolus of anesthetic, the anesthesiologist recompresses the foot pedal 120, thereby contracting the master cylinder 92, extending the slave cylinder 48, contracting the syringe 16, and injecting the anesthetic into the patient 14 through the needle 12.

If, after fully compressing the foot pedal 120 and master cylinder 90, an insufficient volume of anesthetic has been injected, the anesthesiologist can inject more anesthetic using one of two methods.

Using the first method, the anesthesiologist sequentially partially releases and recompresses the foot pedal 120. Partially releasing the foot pedal 120 allows the resilient member 122 to partially uncompress the foot pedal 120. Because the foot pedal 120 is not completely uncompressed, the valve 132 is not opened so that the syringe 16 cannot extend/aspirate. As the foot pedal 120 is uncompressed, the master cylinder 90 extends. Fluid is sucked into the master cylinder 90 from the reservoir 100 through the check valve 104. However, the check valve 96 prevents fluid from being sucked from the slave cylinder 48 into the master cylinder 90 through the conduits 82, 78. Subsequent recompression of the foot pedal 120 recontracts the master cylinder 90, thereby extending the slave cylinder 48, further retracting the syringe 16, injecting additional anesthetic into the patient through the needle 12, and completing the nerve blockade procedure.

Using the second method, which is preferred, the anesthesiologist sequentially fully releases and recompresses the foot pedal 120. Fully releasing the foot pedal 120 opens the valve 132 and aspirates the syringe 16. The mid-injection aspiration warns the anesthesiologist if the needle 12 has been accidentally repositioned during the initial injection. If blood or other body fluid is aspirated, the anesthesiologist knows that the needle is positioned ineffectively and can reposition the needle 12 accordingly. Once the anesthesiologist has been reassured that the needle 12 is positioned properly, he/she recompresses the foot pedal, thereby recontracting the master cylinder 90, extending the slave cylinder 48 further, and injecting additional anesthetic into the patient through the needle 12.

While in the illustrated embodiment, the hands-free syringe controller 40 and hands-free current generator controller 160 are both foot-actuated, the present invention is not so limited. Foot-actuation is simply one example of hands-free control. The goal of the invention is to enable a single anesthesiologist to control both the current generator 140 and the syringe 16 while leaving both of his/her hands free to manipulate and immobilize the needle 12 within the patient 14. In addition to foot-actuation, numerous other types of hands-free controllers may also be used to control the syringe 16 and current generator 140 without departing from the scope of the present invention. For example, as would be readily appreciated by one skilled in the art, conventional voice actuators may be used to control one or both of the syringe 16 and current generator 140.

While the above-described embodiments illustrate specific orientations of the slave cylinder 48, syringe 16, and master cylinder 90, the positions of the respective pistons/plunger relative to their cylinders/base may be switched without deviating from the scope of the present invention. For example, the slave piston portion 50 may be mounted to and move with the plunger 20 instead of the syringe cylinder/base 22.

The foregoing illustrated embodiments are provided to illustrate the structural and functional principles of the present invention and are not intended to be limiting. To the contrary, the principles of the present invention are intended to encompass any and all changes, alterations and/or substitutions within the spirit and scope of the following claims.

What is claimed is:

1. An apparatus for locating and anesthetizing nerves comprising:
a needle adapted to be connected to a syringe having an anesthetic therein;
an electric current generator operatively connected to the needle to selectively apply an electrical stimulus to the needle;
a hands-free current generator controller operatively connected to the current generator, the current generator controller determining electrical characteristics, including at least an amperage, of the electrical stimulus; and
a hands-free syringe controller adapted to be connected to the syringe to selectively aspirate the syringe and inject the anesthetic through the needle.

2. The apparatus of claim 1, wherein the syringe controller comprises a foot pedal movably connected to a base, the foot pedal having compressed and uncompressed positions relative to the base.

3. The apparatus of claim 2, wherein the foot pedal is pivotally mounted to the base.

4. The apparatus of claim 2, wherein the syringe controller further comprises:
a master hydraulic cylinder having a first cylinder portion mounted to one of the foot pedal and the base and a first piston portion mounted to the other of the foot pedal and the base, the first cylinder portion and first piston portion slidingly engaging each other such that compression of the foot pedal contracts the master cylinder;
a slave hydraulic cylinder having a second cylinder portion adapted to be mounted to one of a syringe base and plunger and a second piston portion adapted to be mounted to the other of the syringe base and plunger, the second cylinder portion and second piston portion slidingly engaging each other; and
a first fluid pathway hydraulically connecting the master cylinder to the slave cylinder such that contraction of the master cylinder extends the slave cylinder, which, in turn, is adapted to contract the syringe and push the anesthetic through the needle.

5. The apparatus of claim 4, wherein the syringe controller further comprises:
a first clamp mounted to the second cylinder and adapted to removably secure the one of the syringe base and plunger to the second cylinder; and
a second clamp mounted to the second piston and adapted to removably secure the other of the syringe base and plunger to the second piston.

6. The apparatus of claim 4, further comprising a pressure gauge operatively connected to the slave cylinder, the pressure gauge displaying the pressure within the slave cylinder.

7. The apparatus of claim 4, further comprising a relief valve operatively connected to the slave cylinder, wherein the relief valve opens when the pressure in the slave cylinder exceeds a predetermined pressure.

8. The apparatus of claim 4, wherein the syringe controller further comprises:
a first resilient member operatively connected between the second piston portion and second cylinder portion, the first resilient member tending to contract the slave cylinder and adapted to extend the syringe;
a fluid reservoir;
a second fluid pathway connecting the slave cylinder to the fluid reservoir;
a first check-valve disposed within the second pathway, the first check-valve only allowing fluid to flow through the second pathway toward the reservoir; and
a second valve disposed within the second pathway in series with the first check valve, the second valve being controlled by the foot pedal such that the second valve opens only when the foot pedal is in the uncompressed position.

9. The apparatus of claim 8, wherein the syringe controller further comprises:
a second resilient member disposed between the base and the foot pedal, the second resilient member tending to force the foot pedal into the uncompressed position;
a third fluid pathway connecting the reservoir to the master cylinder;
a third check-valve disposed within the third pathway, the third check-valve only allowing fluid to flow through the third pathway toward the master cylinder; and
a fourth check-valve disposed within the first pathway, the fourth check-valve only allowing fluid to flow through the first pathway toward the slave cylinder.

10. The apparatus of claim 8, wherein the reservoir is mounted to the base and filled with fluid.

11. The apparatus of claim 10, wherein the fluid is hydraulic fluid.

12. The apparatus of claim 10, wherein the fluid is water.

13. The apparatus of claim 12, wherein the syringe controller further comprises a selectively-openable drain operatively connected to the reservoir permitting water to be drained before transporting the apparatus and to be filled before using the apparatus.

14. The apparatus of claim 2, wherein the current generator controller is foot-operated.

15. The apparatus of claim 14, wherein the current generator controller comprises:
first and second foot switches mounted to the base and operatively connected to the electric current generator, wherein actuation of the first foot switch increases the amperage, and wherein actuation of the second foot switch decreases the amperage.

16. The apparatus of claim 14, wherein, when the current generator is not generating a current, actuation of either of the first and second foot switches turns on the generator.

17. The apparatus of claim 1, wherein the current generator is mounted to the syringe controller.

18. The apparatus of claim 1, further comprising an amperage display operatively connected to the current generator, the amperage display being adapted to display the amperage being output by the current generator.

19. The apparatus of claim 1, further comprising a speaker operatively connected to the current generator to call out audibly the amperage being output by the current generator.

20. The apparatus of claim 1, further comprising a light operatively connected to the current generator, the current generator and light being constructed such that the light flashes each time the electric stimulus is applied to the needle.

21. A foot-actuated syringe controller comprising:
a syringe having a syringe base and a plunger;
a foot pedal base; and
a foot pedal movably connected to the foot pedal base, the foot pedal having actuated and unactuated positions relative to the foot pedal base, the foot pedal being operatively connected to the syringe,
wherein moving the foot pedal into the actuated position moves the plunger toward the syringe base, thereby injecting a fluid out of the syringe, and
wherein releasing the foot pedal into the unactuated position applies a force to the plunger that tends to move the plunger away from the syringe base, thereby aspirating the syringe.

22. A foot-actuated syringe controller according to claim 21, further comprising:
a master hydraulic cylinder having a first cylinder portion mounted to one of the foot pedal and the foot pedal base and a first piston portion mounted to the other of the foot pedal and the foot pedal base, the first cylinder portion and first piston portion slidingly engaging each other such that actuation of the foot pedal contracts the master cylinder,
a slave hydraulic cylinder having a second cylinder portion mounted to one of the syringe base and plunger and a second piston portion mounted to the other of the syringe base and plunger, the second cylinder portion and second piston portion slidingly engaging each other; and
a first fluid pathway hydraulically connecting the master cylinder to the slave cylinder such that contraction of the master cylinder extends the slave cylinder, which, in turn, moves the plunger toward the syringe base.

23. A foot-actuated syringe controller according to claim 22, further comprising:
a first resilient member operatively connected between the second piston portion and second cylinder portion, the first resilient member tending to move the plunger away from the syringe, thereby aspirating the syringe;
a fluid reservoir;
a second fluid pathway connecting the slave cylinder to the fluid reservoir;
a first check-valve disposed within the second pathway, the first check-valve only allowing fluid to flow through the second pathway toward the reservoir; and
a second valve disposed within the second pathway in series with the first check valve, the second valve being controlled by the foot pedal such that the second valve opens only when the foot pedal is in the unactuated position.

24. A foot-actuated syringe controller according to claim 23, further comprising:
a second resilient member disposed between the base and the foot pedal, the second resilient member tending to force the foot pedal into the unactuated position;
a third fluid pathway connecting the reservoir to the master cylinder;
a third check-valve disposed within the third pathway, the third check-valve only allowing fluid to flow through the third pathway toward the master cylinder; and
a fourth check-valve disposed within the first pathway, the fourth check-valve only allowing fluid to flow through the first pathway toward the slave cylinder.
